(19) United States
(12) Patent Application Publication
(10) Pub. No.: US 2017/0049797 A1
(43) Pub. Date: Feb. 23, 2017

(54) COMBINATION THERAPY FOR TREATING HCV INFECTION

(71) Applicant: Boehringer Ingelheim International GmbH, Ingelheim am Rhein (DE)

(72) Inventor: Nezam H. AFDHAL, Charlestown, MA (US)

(21) Appl. No.: 15/307,428

(22) PCT Filed: Apr. 30, 2015
(86) PCT No.: PCT/EP2015/059557

§ 371 Date: Oct. 28, 2016

Related U.S. Application Data
(60) Provisional application No. 61/987,682, filed on May 2, 2014.

(51) Int. Cl. A61K 31/7072; A61K 31/4178; A61K 31/7056; A61K 31/4709

(52) U.S. Cl. CPC A61K 31/7072; A61K 31/4709; A61K 31/4178; A61K 31/7056

(57) ABSTRACT

The present invention relates to therapeutic combinations comprising faldaprevir, sofosbuvir, ledipasvir and, optionally, ribavirin, and methods of using such therapeutic combinations for treating HCV infection in a patient.

### FIELD OF THE INVENTION

The present invention relates to new triple and quadruple therapeutic combinations for treating HCV infection (preferably HCV genotype 1 infection, particularly including subgenotypes 1a and/or 1b) where each compound is administered either together or separately. The present invention also provides kits comprising the therapeutic combinations of the present invention.

### BACKGROUND OF THE INVENTION

Hepatitis C virus (HCV) infection is a global human health problem with approximately 150,000 new reported cases each year in the United States alone. HCV is a single stranded RNA virus, which is the etiological agent identified in most cases of non-A, non-B post-transfusion and post-transplant hepatitis and is a common cause of acute sporadic hepatitis. It is estimated that more than 50% of patients infected with HCV become chronically infected and 20% of those develop cirrhosis of the liver within 20 years.

Several types of interferons, in particular, alfa-interferons are approved for the treatment of chronic HCV, e.g., interferon-alfa-2a (ROFERON®-A), interferon-alfa-2b (INTRON®-A), consensus interferon (INFERGEN®), as well as pegylated forms of these and other interferons like pegylated interferon alfa-2a (PEGASYS®) and pegylated interferon alfa-2b (PEG-INTRON®). Ribavirin, a guanosine analog with broad spectrum activity against many RNA and DNA viruses, has been shown in clinical trials to be effective against chronic HCV infection when used in combination with interferon-alfas (see, e.g., Poynard et al., Lancet 352:1426-1432, 1998; Reichard et al., Lancet 351:83-87, 1998), and this combination therapy has been approved for the treatment of HCV: REBETRON® (interferon alfa-2b plus ribavirin, Schering-Plough); PEGASYS®RBV® (pegylated interferon alfa-2a plus ribavirin combination therapy, Roche); see also Manns et al, Lancet 358:958-965 (2001) and Fried et al., 2002, N. Engl. J. Med. 347:975-982. However, even with this combination therapy the sustained virologic response rate among patients chronically infected with genotype 1 is still at or below 50%.

The interferons require administration by injection, which is a much less preferred mode of administration from the standpoint of patient compliance and convenience. Furthermore, there are significant side-effects typically associated with such therapies. Ribavirin suffers from disadvantages that include teratogenic activity, interference with sperm development, haemolysis, anemia, fatigue, headache, insomnia, nausea and/or anorexia. Interferon alfa, with or without ribavirin, is associated with many side effects. During treatment, patients must be monitored carefully for flu-like symptoms, depression, rashes and abnormal blood cell counts. Patients treated with interferon alfa-2b plus ribavirin should not have complications of serious liver dysfunction and such subjects are only considered for treatment of hepatitis C in carefully monitored settings.

Effective and durable therapies that adequately suppress HCV replication typically require combinations of agents that target different biological mechanisms related to HCV infection.

The compound of Formula (I) depicted below is faldaprevir, known as a selective and potent inhibitor of the HCV NS3 serine protease for the treatment of HCV infection:

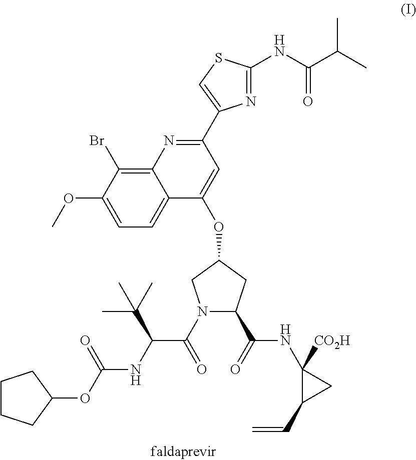 [SMILES: C=C[C@@H]1C[C@]1(NC(=O)[C@@H]1C[C@@H](Oc2cc(-c3csc(NC(=O)C(C)C)n3)nc3c(Br)c(OC)ccc23)CN1C(=O)[C@@H](NC(=O)OC1CCCC1)C(C)(C)C)C(=O)O]

Faldaprevir (CAS Registry No. 801283-95-4) falls within the scope of the acyclic peptide series of HCV inhibitors disclosed in WO 00/09543. Faldaprevir is disclosed specifically as Compound #1055 in WO 2004/103996 (U.S. Pat. No. 7,585,845), and as Compound #1008 in U.S. Pat. No. 7,514,557. Faldaprevir can be prepared according to the general procedures found in the above-cited references. Preferred forms of faldaprevir include the crystalline forms, in particular the crystalline sodium salt form, which can be prepared as described in the examples herein or, for example, in WO 2010/033444, WO 2010/059667, WO 2011/005646, or U.S. Pat. No. 8,232,293.

For the sake of completeness, faldaprevir may also be known by the following alternate depiction of its chemical structure, which is equivalent to the above-described structure:

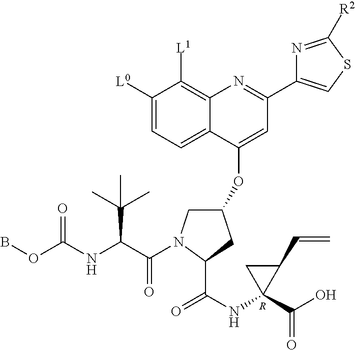

wherein B is

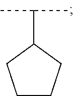

L⁰ is MeO—; L¹ is Br; and R² is

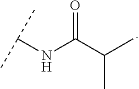

The compound of Formula (II) depicted below is sofosbuvir, which is known as a selective and potent nucleosidic inhibitor of the HCV NS5B RNA-dependent RNA polymerase for the treatment of HCV infection:

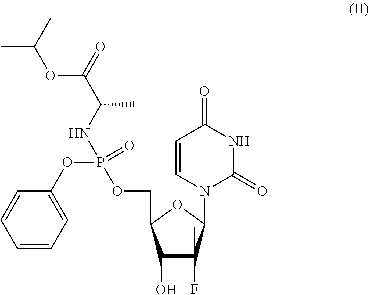

Sofosbuvir (CAS Registry No. 1190307-88-0; GS 7977), falls within the scope of the HCV polymerase inhibitors disclosed in WO 2008/121634 and WO 2010/135569 and one or more of the following U.S. Pat. Nos. 7,964,580; 8,334,270; 8,580,765; 8,618,076; 8,633,309; 8,563,530; 8,629,263 and 8,642,756. Sofosbuvir is disclosed specifically as Example #25 in U.S. Pat. No. 7,964,580. Sofosbuvir and pharmaceutical formulations thereof can be prepared according to the general procedures found in the above-cited references. Sofosbuvir has been approved and commercialized in the US for the treatment of HCV infection.

The compound of Formula (III) depicted below is ledipasvir, which is known as a selective and potent inhibitor of the HCV non-structural 5A (“NS5A”) and therefore useful for the treatment of HCV infection:

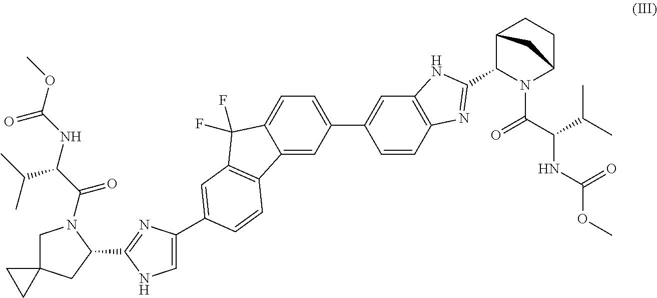 [SMILES: COC(=O)N[C@H](C(=O)N1CC2(CC2)C[C@H]1c1nc(-c2ccc3c(c2)C(F)(F)c2ccc(-c4ccc5nc([C@@H]6[C@@H]7CC[C@@H](C7)N6C(=O)[C@@H](NC(=O)OC)C(C)C)[nH]c5c4)cc2-3)c[nH]1)C(C)C]

Ledipasvir (CAS Registry No. 1256388-51-8; GS-5885), falls within the scope of the HCV NS5A inhibitors disclosed in WO 2010/132601 and WO 2013/184702 and the following U.S. Pat. Nos. 8,088,368; 8,273,341 and 8,575,118. Ledipasvir and pharmaceutical formulations thereof can be prepared according to the general procedures found in the above-cited references.

WO 2011/072370 generally discloses the use of a combination of faldaprevir and one or more additional HCV inhibitors for the treatment of HCV infection.

WO 2013/040492 generally discloses the use of a combination of sofosbuvir and ledipasvir, and optionally additional anti-HCV agents, for the interferon-free treatment of HCV infection. See Compounds 6 (ledipasvir) and 10 (sofosbuvir) in WO 2013/040492.

In general, there is a continuing need for new anti-HCV therapies that are more effective (such as e.g. having broader effectiveness against a wide range of HCV subtypes and IL28B genotypes and/or in patients failing prior anti-HCV therapy), produce less unwanted side-effects and/or reduce the potential for viral rebound. Each of the above referenced compounds is available for oral administration. There is a need for easier to administer direct acting antivirals and all-oral therapies.

Combining multiple direct acting antivirals (DAA) can achieve sustained viral clearance of HCV. HCV differs from some other viruses (such as HIV) in that the viral genome is not archived through a long lived intermediate and the turnover of viral RNA genome in an infected cell is very rapid. The emergence of different classes of HCV DAAs has also revealed that no single DAA alone is sufficient to eliminate the virus from an infected patient and that combinations of different agents are essential to achieve SVR. One class of HCV compounds, the nucleotide inhibitors such as sofosbuvir, has stood out in HCV drug development as possessing a higher barrier to resistance, and requires fewer DAA combination partners to achieve SVR. Sofosbuvir and only one other DAA, either a PI or NS5A inhibitor, can achieve >90% SVR in infected patients following 8-12 weeks of treatment.

A recent exploratory clinical trial, the NIH SYNERGY study, indicates that the sofosbuvir-based 2 DAA regimen may not represent the optimal treatment regimen. Although a sofosbuvir-based 2 DAA regimen has demonstrated high efficacy, still better suppression of viral replication may be achieved with a sofosbuvir-based 3 DAA regimen to enable further shortening of treatment duration. The combination of sofosbuvir with a NS5A inhibitor and a protease inhibitor produced >90% SVR with only 6 weeks of treatment, and contrasts to previous 6 week studies with the sofosbuvir-based 2 DAA regimens that only had 68% SVR.

A model for the clearance of intrahepatic HCV RNA with a half-life of 14 hours (from ex vivo cell culture studies), suggests that if maximal suppression (Emax 100%) of viral replication is achieved, then 28 days of treatment may be sufficient to clear all infectious HCV RNA from an infected patient to achieve SVR.

Four week treatment duration offers substantial benefit in convenience, adherence and cost and represents a unique opportunity for the wide spread use of HCV NS3 protease inhibitors in a highly optimized HCV DAA combination regimen.

### BRIEF SUMMARY OF THE INVENTION

The present invention provides a method of treating HCV infection in a patient comprising the step of administering to the patient an effective amount of a therapeutic combination comprising faldaprevir, sofosbuvir and ledipasvir, or their pharmaceutically acceptable salts, as herein described, and optionally ribavirin. The three or four actives of the combination can be administered simultaneously or separately, as part of a regimen. In another preferred aspect, the combination therapy of the present invention is administered for a treatment duration of 8 weeks or less, preferably 6 weeks or less, even more preferably 4 weeks or less.

### DETAILED DESCRIPTION OF THE INVENTION

### Definitions

As used herein, any reference to “faldaprevir”, “sofosbuvir” or “ledipasvir”, includes each of their pharmaceutically acceptable salts, unless the context indicates otherwise.

The term “about” means within 10%, and more preferably within 5%, of a given value or range. For example, “about 120 mg/day” means from 108 to 132 mg/day, more preferably from 114 to 126 mg/day.

“Ribavirin” refers to 1-β-D-ribofuranosyl-1H-1,2,4-triazole-3-carboxamide, available from ICN Pharmaceuticals, Inc., Costa Mesa, Calif. and is described in the Merck Index, compound No. 8199, Eleventh Edition. Its manufacture and formulation is described in U.S. Pat. No. 4,211,771. Preferred marketed ribavirin products include REBETOL® and COPEGUS®. The term further includes derivatives or analogs thereof, such as those described in U.S. Pat. Nos. 6,063,772, 6,403,564 and 6,277,830. For example, derivatives or analogs include modified ribavirins such as 5′-amino esters, ICN Pharmaceutical's L-enantiomer of ribavirin (ICN 17261), 2′-deoxy derivatives of ribavirin and 3-carboxamidine derivatives of ribavirin, viramidine (previously known as ribamidine) and the like.

The term “pharmaceutically acceptable salt” means a salt of a Compound of formula (1) which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use.

The term includes pharmaceutically-acceptable acid addition salts and pharmaceutically-acceptable base addition salts. Lists of suitable salts are found in, e.g., S. M. Birge et al., J. Pharm. Sci., 1977, 66, pp. 1-19.

The term “pharmaceutically-acceptable acid addition salt” means those salts which retain the biological effectiveness and properties of the free bases and which are not biologically or otherwise undesirable, formed with inorganic acids such as hydrochloric acid, hydrobromic acid, sulfuric acid, sulfamic acid, nitric acid, phosphoric acid, and the like, and organic acids such as acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethane-sulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid, and the like.

The term “pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-acceptable organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like. Particularly preferred organic nontoxic bases are isopropylamine, diethylamine, ethanolamine, trimethylamine, dicyclohexylamine, choline, and caffeine.

The term “therapeutic combination” as used herein means a combination of one or more active drug substances, i.e., compounds having a therapeutic utility. Typically, each such compound in the therapeutic combinations of the present invention will be present in a pharmaceutical composition comprising that compound and a pharmaceutically acceptable carrier. The compounds in a therapeutic combination of the present invention may be administered simultaneously or separately, as part of a regimen. In addition, multiple active drug substances can be combined in a single pharmaceutical dosage form, a so-called “fixed dose combination”. For example, sofosbuvir and ledipasvir are preferably administered together in a fixed dose combination composition containing both sofosbuvir and ledipasvir.

### Embodiments of the Invention

According to a general embodiment, the present invention provides for a method of treating HCV infection in a patient comprising the step of administering to the patient an effective amount of a therapeutic combination comprising (a) faldaprevir, or a pharmaceutically acceptable salt thereof, (b) sofosbuvir, (c) ledipasvir, and optionally (d) ribavirin.

Additional general embodiments include a kit for the treatment of HCV infection comprising: (a) one or more doses of faldaprevir or a pharmaceutically acceptable salt thereof, (b) one or more doses of sofosbuvir, (c) one or more doses of ledipasvir, and optionally ribavirin for the treatment of HCV infection.

In administering the therapeutic combinations of the present invention, each active agent can be administered together at the same time, in a single or multiple dosage forms, or separately at different times in separate dosage administrations. The present invention contemplates and includes all such dosage regimens when administering the triple or quadruple therapeutic combinations as defined herein.

Although this combination therapy is expected to be effective against all HCV genotypes, it is expected to be particularly effective in treating HCV genotype 1 infection, including genotypes (GT) 1a and 1b.

The patient populations to be treated with the combination therapy of the present invention can be further classified into “treatment-naïve” patients, i.e., those patients who have not received any prior treatment for HCV infection, including but not limited to interferon-intolerant or contraindicated patients, and “treatment experienced” patients, i.e., those patients who have undergone prior treatment for HCV. Either of these classes of patients may be treated with the combination therapy of the present invention. A particular class of patients that are preferably treated are those treatment experienced patients that have undergone prior interferon plus ribavirin therapy but are non-responsive to said therapy (herein “non-responders”). Such non-responders include three distinct groups of patients: (1) those who experienced <2×log₁₀ maximum reduction in HCV RNA levels during the first 12 weeks of treatment with interferon plus ribavirin (“null responders”), (2) those who experienced ≧2×log₁₀ maximum reduction in HCV RNA levels during treatment with interferon plus ribavirin but never achieve HCV RNA levels below level of detection (“partial responders”), and (3) those who achieved a virologic response with and during interferon plus ribavirin therapy but had a viral load rebound either during treatment (other than due to patient non-compliance) or after treatment has completed (“relapser”).

According to an alternative embodiment, the present invention provides a method of reducing HCV-RNA levels in a patient in need thereof, comprising the step of administering to said patient a therapeutic combination according to the present invention. Preferably, the method of the present invention reduces the HCV-RNA levels in a patient to a level below the lower limit of quantification (or “BLQ”). A BLQ level of HCV RNA as used in the present invention means a level below 25 International Units (IU) per ml of serum or plasma of a patient as measured by quantitative, multi-cycle reverse transcriptase PCR methodology according to the WHO international standard (Saladanha J, Lelie N and Heath A, Establishment of the first international standard for nucleic acid amplification technology (NAT) assays for HCV RNA. WHO Collaborative Study Group. Vox Sang 76:149-158, 1999). Such methods are well known in the art. In a preferred embodiment, the method of the present invention reduces the HCV-RNA levels in a patient to less than 25 IU per ml of serum or plasma as a result of the treatment. In another embodiment the method of the present invention reduces the HCV-RNA levels in a patient to less than a detectable level as a result of the treatment.

The usual duration of the treatment for standard interferon plus ribavirin therapy is at least 48 weeks for HCV genotype 1 infection, and at least 24 weeks for HCV genotypes 2 and 3. However, with the triple and quadruple combination therapies of the present invention it may be possible to have a much shorter duration of treatment. With the combination therapy of the present invention it is expected that the duration of traditional HCV therapy can be significantly reduced to less than 12 weeks, preferably 8 weeks or less, more preferably 6 weeks or less, even more preferably 4 weeks or less. The time period for different HCV genotypes, e.g. HCV genotypes 2, 3, 4, 5 or 6 is expected to be similar.

The first component of the therapeutic combination, namely, faldaprevir or a pharmaceutically acceptable salt thereof is comprised in a composition. Such a composition comprises faldaprevir, or a pharmaceutically acceptable salt thereof, and a pharmaceutically acceptable adjuvant or carrier. Typical pharmaceutical compositions that may be used for faldaprevir, or a pharmaceutically acceptable salt thereof, are as described in U.S. Pat. No. 7,514,557. Further specific examples of compositions are as set forth in the examples section below. A particularly preferred from of faldaprevir to be used is the sodium salt form.

In general, the faldaprevir or a pharmaceutically acceptable salt thereof may be administered at a dosage of at least 80 mg/day (in single or divided doses). Additional embodiments for dosage amounts and ranges may include (in single or divided doses):
(a) from about 80 mg/day to about 240 mg/day
(b) from about 120 mg/day to about 240 mg/day
(c) about 80 mg/day
(d) about 120 mg/day
(e) about 240 mg/day

Although faldaprevir may be administered in single or divided daily doses, once a day administration (QD) of the daily dose is preferred. A particularly preferred faldaprevir dosage is 120 mg/day (QD). As the skilled artisan will appreciate, however, lower or higher doses than those recited above may be required. Specific dosage and treatment regimens for any particular patient will depend upon a variety of factors, including the age, body weight, general health status, sex, diet, time of administration, rate of excretion, drug combination, the severity and course of the infection, the patient's disposition to the infection and the judgment of the treating physician. In general, the compound is most desirably administered at a concentration level that will generally afford antivirally effective results without causing any harmful or deleterious side effects.

In another embodiment according to the invention, a loading dose amount of faldaprevir is administered for the first administration dose of the treatment. The loading dose amount is higher than the dose amount administered for subsequent administrations in the treatment. Preferably, the loading dose amount is about double in quantity, by weight, of the amount in subsequent administrations in the treatment. For example, in one embodiment, the first dose of faldaprevir administered at dosage of about 240 mg and subsequent doses of faldaprevir are administered at a dosage of about 120 mg. In another embodiment, the first dose of faldaprevir administered at a dosage of about 480 mg and subsequent doses of faldaprevir are administered at a dosage of about 240 mg. In another embodiment, the first dose of faldaprevir administered is at a dosage of about 960 mg and subsequent doses of faldaprevir are administered at a dosage of about 480 mg.

By using this loading dose concept, a clear advantage is that it is thereby possible to achieve steady state levels of active drug in the patient's system earlier than would otherwise be achieved. The blood level achieved by using a doubled loading dose is the same as would be achieved with a double dose but without the safety risk attendant to the subsequent continuous administration of a double dose. By reaching the targeted steady state level of active drug earlier in therapy also means that there less possibility of insufficient drug pressure at the beginning of therapy so that resistant viral strains have a smaller chance of emerging.

The second component of the therapeutic combination, namely, sofosbuvir, or a pharmaceutically acceptable salt thereof, is comprised in a composition. Such a composition comprises sofosbuvir, or a pharmaceutically acceptable salt thereof, and a pharmaceutically acceptable adjuvant or carrier. Typical pharmaceutical compositions that may be used for sofosbuvir are as described in U.S. Pat. No. 7,964,580 and WO 2013/040492.

In general, the sofosbuvir may be administered at dosage amounts and in dose ranges that may include (in single or divided doses):
(a) from about 300 mg/day to about 500 mg/day
(b) from about 350 mg/day to about 450 mg/day
(c) about 300 mg/day
(d) about 400 mg/day
(e) about 500 mg/day

Although sofosbuvir may be administered in single or divided daily doses, once a day (QD) dosing is preferred. Particularly preferred sofosbuvir dosage includes 400 mg QD. As the skilled artisan will appreciate, however, lower or higher doses than those recited above may be required. Specific dosage and treatment regimens for any particular patient will depend upon a variety of factors, including the age, body weight, general health status, sex, diet, time of administration, rate of excretion, drug combination, the severity and course of the infection, the patient's disposition to the infection and the judgment of the treating physician. In general, the compound is most desirably administered at a concentration level that will generally afford antivirally effective results without causing any harmful or deleterious side effects.

The third component of the therapeutic combination, ledipasvir, is also comprised in a pharmaceutical composition. Such a composition comprises ledipasvir, or a pharmaceutically acceptable salt thereof, and a pharmaceutically acceptable adjuvant or carrier. Typical pharmaceutical compositions that may be used for ledipasvir are as described in WO 2010/132601.

In general, ledipasvir may be administered at dosage amounts and in dose ranges that may include (in single or divided doses):
(a) from about 80 mg/day to about 100 mg/day
(b) about 80 mg/day
(c) about 90 mg/day
(d) about 100 mg/day

Although ledipasvir may be administered in single or divided daily doses, once a day administration (QD) of the daily dose is preferred. A particularly preferred ledipasvir dosage is 90 mg/day (QD). As the skilled artisan will appreciate, however, lower or higher doses than those recited above may be required. Specific dosage and treatment regimens for any particular patient will depend upon a variety of factors, including the age, body weight, general health status, sex, diet, time of administration, rate of excretion, drug combination, the severity and course of the infection, the patient's disposition to the infection and the judgment of the treating physician. In general, the compound is most desirably administered at a concentration level that will generally afford antivirally effective results without causing any harmful or deleterious side effects.

In a particular embodiment of the invention, the sofosbuvir and ledipasvir are incorporated together in a single dosage form, as a so-called “fixed dose combination” product. In one example, the product contains 400 mg sofosbuvir and 90 mg ledipasvir in a single dosage form, preferably a single tablet that is administered once per day.

The optional fourth component of the therapeutic combination, namely ribavirin, is comprised in a pharmaceutical composition. Typically, such compositions comprise ribavirin and a pharmaceutically acceptable adjuvant or carrier and are well known in the art, including in a number of marketed ribavirin formulations. Formulations comprising ribavirin are also disclosed, e.g., in U.S. Pat. No. 4,211,771.

The types of ribavirin that may be used in the combination are as outlined hereinabove in the definitions section. In one preferred embodiment, the ribavirin is either REBETOL® or COPEGUS® and they may be administered at their labeled dosage levels indicated for interferon plus ribavirin combination therapy for the treatment of HCV infection. Of course, with the triple combination therapy of the present invention it may be possible to use a lower dosage of ribavirin, e.g., lower than is used the current standard interferon plus ribavirin therapy, while delivering the same or better efficacy than the current standard therapy with less side-effects usually associated with such therapy.

According to various embodiments, the ribavirin may be administered at dosages of (in single or divided doses):
(a) between about 200 mg/day to about 1800 mg/day;
(b) between about 800 mg/day to about 1200 mg/day;
(c) between about 1000 mg/day to about 1200 mg/day;
(d) about 1000 mg/day;
(e) about 1200 mg/day;
(f) between about 400 mg/day to about 800 mg/day
(g) about 400 mg/day
(h) about 600 mg/day
(i) about 800 mg/day

According to one embodiment, the ribavirin composition comprises ribavirin in a formulation suitable for dosing once a day or twice daily. For example, if a therapeutic combination comprises about 1000 mg/day dosage of ribavirin, and a dosing of two times a day is desired, then the therapeutic combination will comprise ribavirin in a formulation, e.g., a tablet, containing, e.g., about 200 mg of ribavirin, with the first dose of 600 mg (or 400 mg), followed by a second dose of 400 mg (or 600 mg) at least 6 hours apart.

In addition to the above-mentioned three or four components of the combination, additional anti-HCV agents may be added and used with the combination therapy of the present invention. Suitable additional anti-HCV agents are well known in the art, and, for example, are described in WO 2004/103996, WO 2005/080388, WO 2010/065674, and WO2013/123092.

### Additional Sub-Embodiments

For example, in one embodiment the present invention contemplates a method of treating hepatitis C viral (HCV) infection comprising the step of administering to the patient a therapeutic combination comprising:
(a) faldaprevir or a pharmaceutically acceptable salt thereof at a dosage between about 80 mg/day and about 240 mg/day;
(b) sofosbuvir at a dosage between about 300 mg/day to about 500 mg/day;
(c) ledipasvir at a dosage between about 80 mg/day and about 100 mg/day;
(d) optionally ribavirin at a dosage of between about 200 mg/day to about 1800 mg/day.

In another embodiment the present invention contemplates a method of treating hepatitis C viral (HCV) infection in a patient comprising the step of administering to the patient a therapeutic combination comprising:
(a) faldaprevir or a pharmaceutically acceptable salt thereof at a dosage between about 120 mg/day to about 240 mg/day;
(b) sofosbuvir at a dosage between about 350 mg/day to about 450 mg/day;
(c) ledipasvir at a dosage between about 80 mg per day and about 100 mg per day;
(d) optionally ribavirin at a dosage of between about 1000 mg/day to about 1200 mg/day.

In another embodiment the present invention contemplates a method of treating hepatitis C viral (HCV) infection in a patient comprising the step of administering to the patient a therapeutic combination comprising:
(a) faldaprevir or a pharmaceutically acceptable salt thereof at a dosage of 120 mg/day;
(b) sofosbuvir at a dosage of 400 mg/day; and
(c) ledipasvir at a dosage of 90 mg per day.

In another embodiment, the present invention contemplates a method of treating hepatitis C viral (HCV) infection in a patient comprising the step of administering to the patient a combination of:
(a) a single dosage form containing 400 mg of sofosbuvir and 90 mg of ledipasvir; and
(b) a single dosage form containing 120 mg faldaprevir or its pharmaceutically acceptable salt;
   and, preferably, wherein the combination of the two dosage forms is administered once per day.

Further embodiments include any of the above-mentioned embodiments wherein the faldaprevir is administered in the form of its sodium salt. For example, an additional embodiment is directed to a method of treating hepatitis C viral (HCV) infection in a patient comprising the step of administering to the patient a combination of:
(a) a single dosage form containing 400 mg of sofosbuvir and 90 mg of ledipasvir; and
(b) a single dosage form containing 120 mg faldaprevir in the form of its sodium salt;

and, preferably, wherein the combination of the two dosage forms is administered once per day.

Further embodiments include any of the above-mentioned embodiments, and where:
(a) the therapy is a triple combination therapy including administration of faldaprevir, sofosbuvir and ledipasvir; or
(b) the therapy is a quadruple combination therapy including administration of faldaprevir, sofosbuvir, ledipasvir and ribavirin.

Further embodiments include any of the above-mentioned embodiments, and where:
(a) the HCV infection is genotype 1 and the patient is a treatment-naïve patient; or
(b) the HCV infection is genotype 1 and the patient is a treatment-experienced patient.

Further embodiments include any of the above-mentioned embodiments, and where the HCV infection is genotype 1a, and in a further embodiment the HCV infection is genotype 1a and the patient has an “unfavorable” IL28B subgenotype (i.e. having non-CC genotype of SNP rs12979860 or a non-TT genotype of SNP rs 8099917).

Further embodiments include any of the above-mentioned embodiments, and where the faldaprevir is administered once a day, sofosbuvir is administered once a day, ledipasvir is administered once a day and the ribavirin, if included in the therapy, is administered twice a day.

Further embodiments include any of the above-mentioned embodiments and where the loading dose concept in used for faldaprevir, e.g., the first dose of faldaprevir administered is double in quantity to the subsequent doses.

Further embodiments include any of the above-mentioned embodiments, and where the therapeutic regimen of the present invention is administered to the patient for less than 12 weeks, preferably 8 weeks or less, more preferably 6 weeks or less, and more preferably 4 weeks or less.

With respect to the triple or quadruple combination therapies of the present invention, the present invention contemplates and includes all combinations of the various preferred embodiments and sub-embodiments as set forth herein.

In an additional embodiment, there is limited or no emergence of viral resistance during the combination therapy of the present invention

### EXAMPLES

### I. Methods for Preparing Faldaprevir

Methods for preparing amorphous faldaprevir and a general description of pharmaceutically acceptable salt forms can be found in U.S. Pat. Nos. RE 40,525, 7,514,557 and 7,585,845. Methods for preparing additional forms of faldaprevir, in particular the crystalline sodium salt form, can be found in U.S. Pat. No. 8,232,293.

### II. Formulations of Faldaprevir

One example of a pharmaceutical formulation of faldaprevir include an oral solution formulation as disclosed in WO 2010/059667. Additional examples include capsules containing a lipid-based liquid formulation, as disclosed in WO 2011/005646.

### III. Methods for Preparing Sofosbuvir, Ledipasvir and Formulations Thereof

Methods for preparing sofosbuvir, ledipasvir and pharmaceutical formulations thereof can be found in the above-cited patent publications relating to these compounds. See the patent publications cited in the Background of the Invention section above.

### IV. Data/Results

The recently published results from the NIH SYNERGY trial, combining sofosbuvir, ledipasvir and a protease inhibitor, demonstrating high effectiveness with a 6 week treatment duration, suggest that the combination therapy of the present invention would likewise be an effective HCV treatment option. The published results are reproduced below:

Combination Oral, Hepatitis C Antiviral Therapy for 6 or 12 Weeks: Final Results of the SYNERGY Trial*
*Reference: Kohli et al., Abstract 27LB, Conference on Retroviruses and Opportunistic Infections (CROI), Mar. 3-6, 2014, Boston, Mass.

Background: Combining multiple directly acting antiviral agents (DAA) is a plausible approach to reduce the duration of therapy require to cure HCV infection. In this study treatment regimens targeting multiple stages of HCV replication were evaluated for safety and efficacy in a historically difficult to treat population.

Methodology: Sixty HCV mono-infected, treatment naïve, GT-1, patients were consecutively enrolled into 3 arms of a phase 2 prospective cohort study and received: Arm A—sofosbuvir with ledipasvir (400 mg/90 mg respectively once daily in a fixed dose combination (FDC)) for 12 weeks, Arm B—FDC+GS-9669 (500 mg/day), a non nucleoside NS5B inhibitor for 6 weeks, or Arm C—FDC+GS-9451 (80 mg/day), an HCV protease inhibitor for 6 weeks. Serial measurements of safety parameters, virologic (HCV RNA by Roche Taqman PCR and Abbott assay; deep sequencing of baseline mutations) and host correlates (intrahepatic and peripheral by flow cytometry) were performed.

Results: Patients enrolled were predominantly African American (88%), male (72%), infected with GT-1a (70%), had a high HCV VL (>800 k) (70%) with an IL28B non-CC haplotype (82%). Baseline demographics were similar between patients across arms and 35%, 25% and 25% of subjects on Arm A, B and C, respectively, had stage 3 liver fibrosis. No patients with cirrhosis were included in Arm B or C. The end of treatment response (HCV RNA <LLOQ) was 100%, 75% and 95% of subjects in Arms A, B and C respectively using a more sensitive HCV assay with lower limit of quantification of 12 IU/mL. Using an HCV RNA assay with a lower limit of quantification of <43 IU/mL 100% of patients on all arms were suppressed at EOT. 100%, 90% and 95% of patients in Arm A, B and C respectively achieved SVR12. One patient in Arm B experienced viral relapse. One patient on Arms A and B each missed their SVR12 visit. The combination was well tolerated with no grade 4 adverse events or drug discontinuations.

Conclusions: In this inner city patient population addition of a third antiviral agent allowed successful eradication of HCV in 6 weeks in a difficult to treat patient population. This study presents a new paradigm of combination therapy to reduce HCV treatment duration, which may be vital in the treatment and eradication of HCV globally.

1-17. (canceled)

18. A therapeutic combination comprising:
(a) faldaprevir, or a pharmaceutically acceptable salt thereof;
(b) sofosbuvir, and
(c) ledipasvir,
for use in a method of treating hepatitis C viral (HCV) infection in a human.

19. The therapeutic combination according to claim 18 for use in a method of treating an HCV genotype 1 infection.

20. The therapeutic combination according to claim 18 for use in a method of treating a treatment-naive patient.

21. The therapeutic combination according to claim 18 for use in a method of treating a treatment experienced patient.

22. The therapeutic combination according to claim 18 reducing the HCV-RNA levels to a level below 25 International Units (IU) per ml of serum or plasma.

23. The therapeutic combination according to claim 18 comprising faldaprevir or a pharmaceutically acceptable salt thereof at a dosage between 80 mg and 240 mg.

24. The therapeutic combination according to claim 18 claims comprising sofosbuvir at a dosage between 300 mg and 500 mg.

25. The therapeutic combination according to claim 18 comprising ledipasvir at a dosage between 80 mg and 100 mg.

26. The therapeutic combination according to claim 18 for use in administering:
(a) faldaprevir, or a pharmaceutically acceptable salt thereof, at a dosage of 120 mg;
(b) sofosbuvir at a dosage of 400 mg; and
(c) ledipasvir at a dosage of 90 mg
to treat an hepatitis C viral (HCV) infection in a human.

27. The therapeutic combination according to claim 18, wherein faldaprevir is in the form of its sodium salt.

28. The therapeutic combination according to claim 18, comprising faldaprevir or a pharmaceutically acceptable salt thereof, sofosbuvir, and ledipasvir.

29. The therapeutic combination according to claim 28, wherein faldaprevir, or its pharmaceutically acceptable salt, is comprised in a separate dosage form.

30. The therapeutic combination according to claim 18 further comprising ribavirin.

31. The compound faldaprevir or a pharmaceutically acceptable salt thereof, for use in a method for the treatment of HCV infection in a human, wherein the faldaprevir or pharmaceutically acceptable salt thereof is used together with sofosbuvir and ledipasvir.

32. The compound sofosbuvir for use in a method for the treatment of HCV infection in a human, wherein the sofosbuvir is used together with faldaprevir or a pharmaceutically acceptable salt thereof and ledipasvir.

33. The compound ledipasvir for use in a method for the treatment of HCV infection in a human, wherein the ledipasvir is used together with faldaprevir or a pharmaceutically acceptable salt thereof and sofosbuvir.

34. The compound according to claim 31, wherein ribavirin is additionally used.

35. The compound according to claim 32, wherein ribavirin is additionally used.

36. The compound according to claim 33, wherein ribavirin is additionally used.
